(19) United States
(12) Patent Application Publication
(10) Pub. No.: US 2014/0309481 A1
(43) Pub. Date: Oct. 16, 2014

(54) ROTARY PUMP WITH LEVITATED IMPELLER HAVING THRUST BEARING FOR IMPROVED STARTUP

(71) Applicant: THORATEC CORPORATION, Pleasanton, CA (US)

(72) Inventors: Alexander L. Medvedev, Ann Arbor, MI (US); Masamichi Yanai, Ann Arbor, MI (US)

(73) Assignee: THORATEC CORPORATION, Pleasanton, CA (US)

(21) Appl. No.: 13/860,569

(22) Filed: Apr. 11, 2013

(51) Int. Cl. A61M 1/10

(52) U.S. Cl. CPC A61M 1/1012
USPC 600/16

(57) ABSTRACT

A rotary blood pump comprises an impeller in a pump housing with a pumping chamber between first and second walls. The impeller operates in a levitated position spaced from the first and second walls in response to hydrodynamic forces which are boosted by hydrodynamic bearing features in the walls. At least one of the impeller or the walls includes at least one mechanical thrust bearing extending between the impeller and each of the walls, wherein the mechanical thrust bearing is configured such that when the impeller is not being held in the levitated position by the hydrodynamic forces then the mechanical thrust bearing is engaged to maintain a predetermined separation between the hydrodynamic bearing features and the impeller. The mechanical thrust bearing is configured such that when the impeller is being held in the levitated position by the hydrodynamic forces then the mechanical thrust bearing is unengaged.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to centrifugal pumping devices for circulatory assist and other uses, and, more specifically, to an improved startup of a magnetically-levitated impeller that avoids excessive wear of the impeller against the housing before levitation is obtained.

Many types of circulatory assist devices are available for either short term or long term support for patients having cardiovascular disease. For example, a heart pump system known as a left ventricular assist device (LVAD) can provide long term patient support with an implantable pump associated with an externally-worn pump control unit and batteries. The LVAD improves circulation throughout the body by assisting the left side of the heart in pumping blood. One such system is the DuraHeart® LVAS system made by Terumo Heart, Inc., of Ann Arbor, Mich. The DuraHeart® system employs a centrifugal pump with a magnetically levitated impeller to pump blood from the left ventricle to the aorta. The impeller acts as a rotor of an electric motor in which a rotating magnetic field generated in the pump housing (from either the coils of a multiphase stator or a spinning rotor carrying permanent magnets) couples with the impeller which is rotated at a speed appropriate to obtain the desired blood flow through the pump.

The centrifugal pump employs a sealed pumping chamber. By levitating the impeller within the chamber when it rotates, turbulence in the blood is minimized. The spacing between the impeller and chamber walls minimizes pump-induced hemolysis and thrombus formation. The levitation is obtained by the combination of a magnetic bearing and a hydrodynamic bearing. For the magnetic bearing, the impeller typically employs upper and lower plates having permanent magnetic materials for interacting with a magnetic field applied via the chamber walls. For example, a stationary magnetic field may be applied from the upper side of the pump housing to attract the upper plate while a rotating magnetic field from the lower side of the pump housing (to drive the impeller rotation) attracts the lower plate. The hydrodynamic bearing results from the action of the fluid between the impeller and the chamber walls while pumping occurs. Grooves may be placed in the chamber walls to enhance the hydrodynamic bearing (as shown in U.S. Pat. No. 7,470,246, issued Dec. 30, 2008, titled “Centrifugal Blood Pump Apparatus,” which is incorporated herein by reference). The magnetic and hydrodynamic forces cooperate so that the impeller rotates at a levitated position within the pumping chamber.

Prior to starting rotation of the impeller, the axial forces acting on it are not balanced. Magnetic attraction causes the impeller to rest against one of the upper or lower chamber walls. In many pump designs, it is possible for the impeller to be arbitrarily resting against either one of the walls. When rotation begins, the rubbing of the impeller against the chamber wall can cause undesirable mechanical wear of the impeller and/or wall. The amount of wear is proportional to the rotation angle traversed until the impeller lifts off of the pump housing and to the normal force between the impeller and housing.

In one typical startup sequence of the prior art, the stator coils are energized to produce a strong, stationary magnetic field that rotates the impeller into alignment with a known phase angle. When the impeller moves during alignment, it typically overshoots the desired position due to the strong field and then it oscillates around the desired position until the motion dampens out. Much mechanical wear can occur during this step. Once in the aligned position, the pump motor accelerates the impeller until the hydrodynamic bearing forces separate it from the chamber wall. However, the normal force can be high before separation occurs, further increasing the wear. Additional wear also occurs when pump operation is stopped since the impeller speed will typically continue to coast down after the lift from the hydrodynamic forces become insufficient to maintain levitation.

In order to handle the inherent wear and abrasion problems, conventional pumps have employed materials with a high hardness or have applied special coatings such as a fluorinated coating or a diamond-like carbon coating. However, harder materials have lower manufacturability, resulting in more costly manufacturing as well as higher development costs. Similarly, the use of a coating results in higher costs and time for both manufacturing and development. It would be desirable to employ softer biocompatible materials such as titanium or a titanium alloy without suffering from excessive wear.

### SUMMARY OF THE INVENTION

In one aspect of the invention, a rotary blood pump comprises a pump housing with a pumping chamber between first and second walls, and an impeller disposed in the pumping chamber. The impeller is configured to operate in a levitated position spaced from the first and second walls in response to hydrodynamic forces that urge the impeller into the levitated position. A portion of the first and second walls includes hydrodynamic bearing features for increasing the hydrodynamic forces. At least one of the impeller or the walls includes at least one mechanical thrust bearing extending between the impeller and each of the walls, wherein the mechanical thrust bearing is configured such that when the impeller is not being held in the levitated position by the hydrodynamic forces then the mechanical thrust bearing is engaged to maintain a predetermined separation between the hydrodynamic bearing features and the impeller. The mechanical thrust bearing is configured such that when the impeller is being held in the levitated position by the hydrodynamic forces then the mechanical thrust bearing is unengaged.

### BRIEF DESCRIPTION OF THE DRAWINGS

FIG. 1 is a diagram of a circulatory assist system as one example of an implantable pump employing the present invention.

FIG. 2 is an exploded, perspective view of a rotary pump housing and impeller.

FIG. 3 is a cross section showing an impeller levitated to a centered position within a pumping chamber.

FIG. 4 is a plan view of a chamber wall having hydrodynamic bearing features.

FIG. 5 is a side, cross-sectional view showing hydrodynamic bearing forces levitating the impeller.

FIG. 6 is a side, cross-sectional view showing a stopped impeller resting on a chamber wall in the absence of hydrodynamic bearing forces.

FIG. 7 is a side, cross-sectional view showing a mechanical thrust bearing on the pump housing.

FIG. 8 is a side, cross-sectional view showing a mechanical thrust bearing on the impeller.

FIG. 9 is a top view of the impeller showing a plurality of raised bumps forming the mechanical thrust bearing.

FIG. 10 is top view of the impeller showing the mechanical thrust bearing as an arcuate rib.

FIG. 11 is a cross section of the impeller of FIG. 10.

FIG. 12 is a side, cross-sectional view showing an alternative embodiment of a mechanical thrust bearing using a ramp surface.

FIG. 13 is a cross-sectional view of a pump having a cylindrically-shaped impeller and pumping chamber, with a mechanical thrust bearing formed as raised bumps.

FIG. 14 is a cross-sectional view of a pump having a cylindrically-shaped impeller and pumping chamber, with a mechanical thrust bearing formed as ramped surfaces.

### DETAILED DESCRIPTION OF PREFERRED EMBODIMENTS

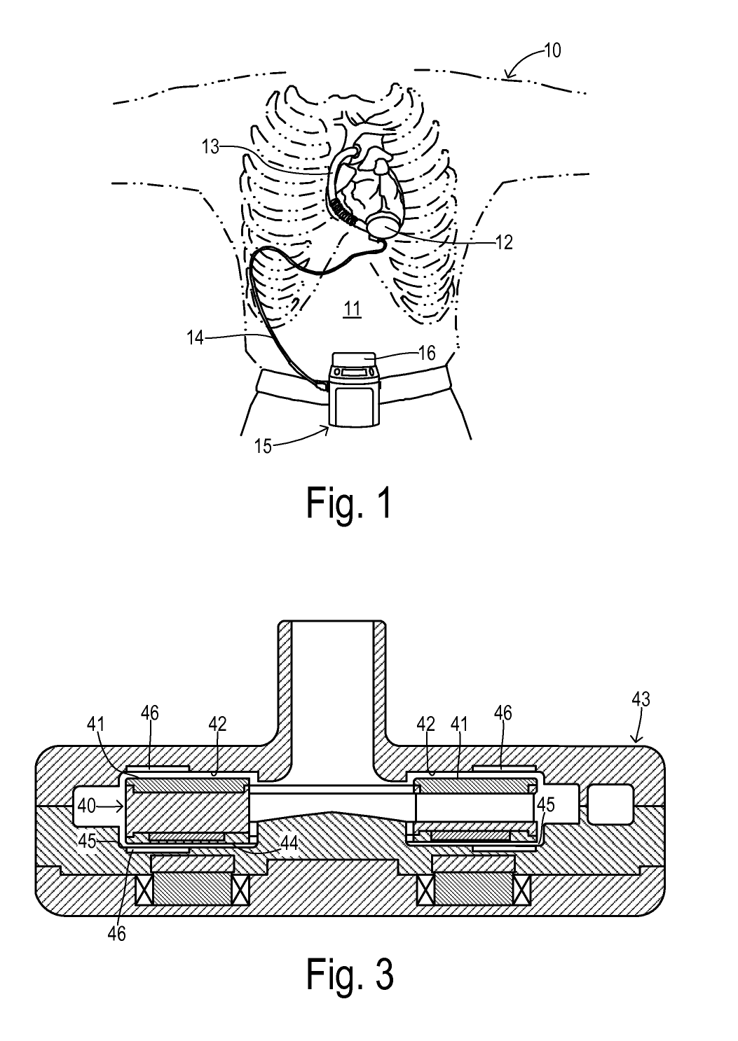

Referring to FIG. 1, a patient 10 is shown in fragmentary front elevational view. Surgically implanted either into the patient's abdominal cavity or pericardium 11 is the pumping unit 12 of a ventricular assist device. An inflow conduit (on the hidden side of unit 12) pierces the heart to convey blood from the patient's left ventricle into pumping unit 12. An outflow conduit 13 conveys blood from pumping unit 12 to the patient's aorta. A percutaneous power cable 14 extends from pumping unit 12 outwardly of the patient's body via an incision to a compact control unit 15 worn by patient 10. Control unit 15 is powered by a main battery pack 16 and/or an external AC power supply and an internal backup battery. Control unit 15 includes a commutator circuit for driving a motor stator within pumping unit 12.

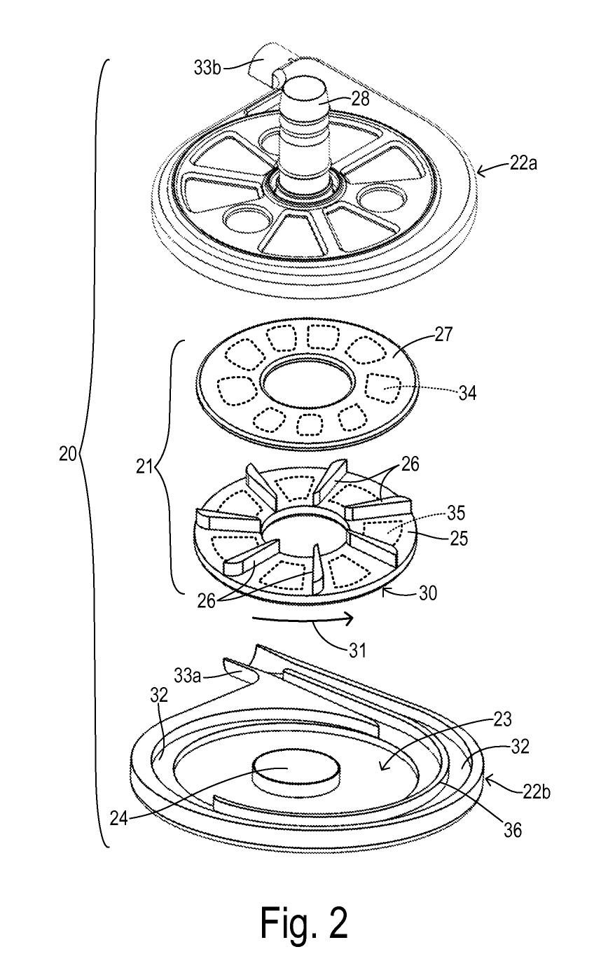

FIG. 2 shows a centrifugal pump unit 20 having an impeller 21 and a pump housing having upper and lower halves 22a and 22b. Impeller 21 is disposed within a pumping chamber 23 over a hub 24. Impeller 21 includes a first plate or disc 25 and a second plate or disc 27 sandwiched over a plurality of vanes 26. Second disc 27 includes a plurality of embedded magnet segments 34 for interacting with a levitating magnetic field created by a levitation magnet structure (not shown) disposed against housing 22a. First disc 25 contains embedded magnet segments 35 for magnetically coupling with a rotating magnetic field from a stator assembly (not shown) disposed against housing 22b. Housing 22a includes an inlet 28 for receiving blood from a patient's ventricle and distributing it to vanes 26. Impeller 21 is preferably circular and has an outer circumferential edge 30. By rotatably driving impeller 21 in a pumping direction 31, the blood received at an inner edge of impeller 21 is carried to outer circumferential edge 30 and enters a volute region 32 within pumping chamber 23 at an increased pressure. The pressurized blood flows out from an outlet 33 formed by housing features 33a and 33b. A flow-dividing guide wall 36 may be provided within volute region 32 to help stabilize the overall flow and the forces acting on impeller 21.

The cross section of FIG. 3 shows a similar embodiment wherein an impeller 40 is located at a centered (levitated) position wherein a top surface 41 of impeller 40 is spaced from a first wall 42 of a pump housing 43 and a bottom surface 44 of impeller 40 is spaced from a second wall 45 of pump housing 43. Hydrodynamic bearing forces normally exerted on impeller 40 by the circulating fluid (e.g., blood) are increased by forming hydrodynamic pressure features 46 in walls 42 and 45. Features 46 can be either grooves sunk into the surface or raised wedges extending from the surface.

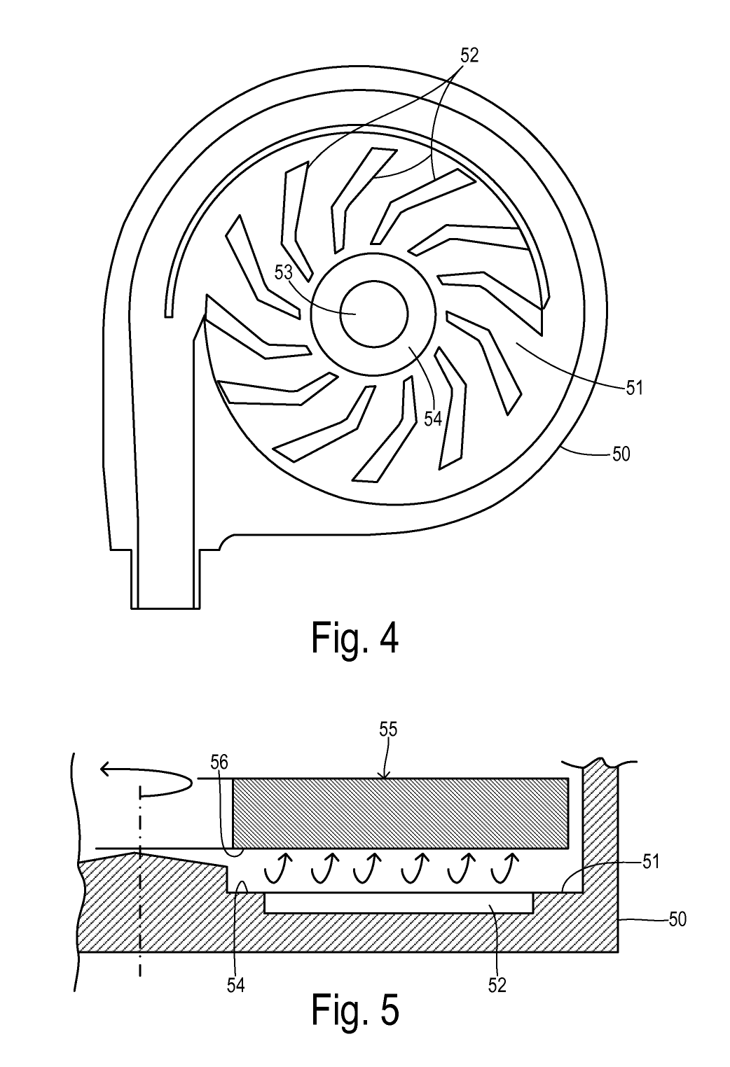

FIG. 4 is an internal view of a rotary pump housing with one side of a housing 50 having a wall 51 forming one side of a pumping chamber. Hydrodynamic bearing features 52 are disposed in wall 51 to add an axial component to the localized flow to create a lift that urges the impeller away from wall 51. The impeller fits over a central hub 53, and there may be a surrounding radial band 54 from which the hydrodynamic bearing features 52 are excluded.

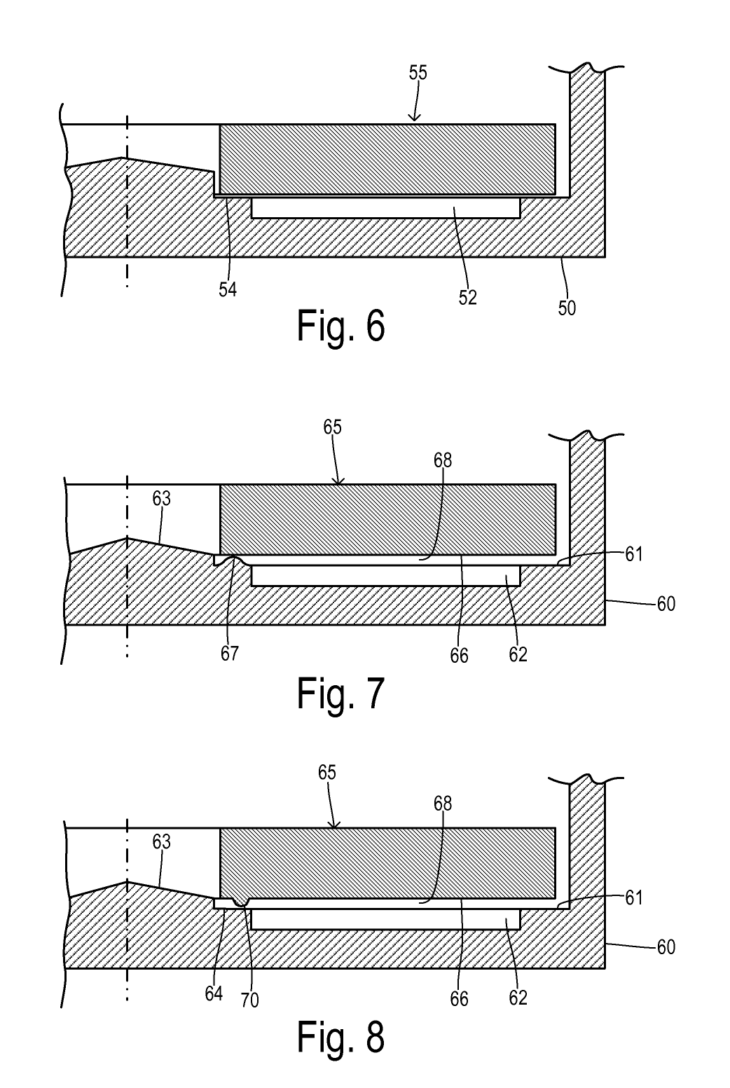

FIG. 5 shows at an exaggerated scale the levitation of an impeller 55 as a result of the hydrodynamic forces enhanced by hydrodynamic bearing features 52. Impeller 55 is lifted away from wall 51 so that a bottom surface 56 of impeller 55 reaches a levitated position away from wall 51. FIG. 6 shows the resting position of impeller 55 when pump operation is stopped. Thus, surfaces 56 and 51 are in contact over a large surface area including radial band 54 and the areas surrounding hydrodynamic bearing grooves 52. Because of the large contacting surface area, significant wear and abrasion can occur prior to achieving levitation by the hydrodynamic bearing forces.

The present invention solves the foregoing problem by the addition of a mechanical thrust bearing between the impeller and the wall of the pumping chamber which is configured so that when the impeller is not being held in the levitated position by the hydrodynamic forces, then the mechanical thrust bearing is engaged to maintain a predetermined separation between the hydrodynamic bearing features and the impeller. Embodiments are shown in which the mechanical thrust bearing may be incorporated into either the impeller or the pump housing.

A first embodiment of the invention is shown in FIG. 7 wherein a pump housing 60 has a wall 61 forming one side of a pumping chamber and has a hydrodynamic bearing feature 62 concentrically around a central hub 63. An impeller 65 has a surface 66 juxtaposed with housing wall 61. FIG. 7 shows impeller 65 in a stopped condition so that no hydrodynamic bearing forces are acting on impeller 65. Instead, it is suspended by a mechanical thrust bearing 67 in the form of a raised bump protruding from wall 61. Thrust bearing 67 supports surface 66 with a predetermined separation 68 between hydrodynamic bearing feature 62 and impeller surface 66. Thus, when pump operation is being started or stopped and an insufficient rotation speed results in insufficient lift to levitate impeller 65 away from mechanical thrust bearing 67, the area of contact between pump housing 60 and impeller 65 is nevertheless greatly reduced to such an extent that friction and abrasion are greatly eliminated. The contact area is so small and the friction so slight that softer materials can be used for the pump component and no coatings are required.

As shown in FIG. 8, the mechanical thrust bearing can also be located on impeller 65. Thus, a raised bump 70 extends from surface 66 of impeller 65 to contact wall 61 of pump housing 65 within a radial band 64 of wall 61 (i.e., away from hydrodynamic bearing features 62). Preferably, radial band 64 is located radially inward from a primary radial band which is occupied by hydrodynamic bearing feature 62 because there is a lower shear rate radially inward of the primary band occupied by features 62.

Although a mechanical thrust bearing is shown only on one side of the impeller in FIGS. 7 and 8, such bearings are preferably located on both sides of the impeller (depending upon whether the impeller can come to a rest position against either side).

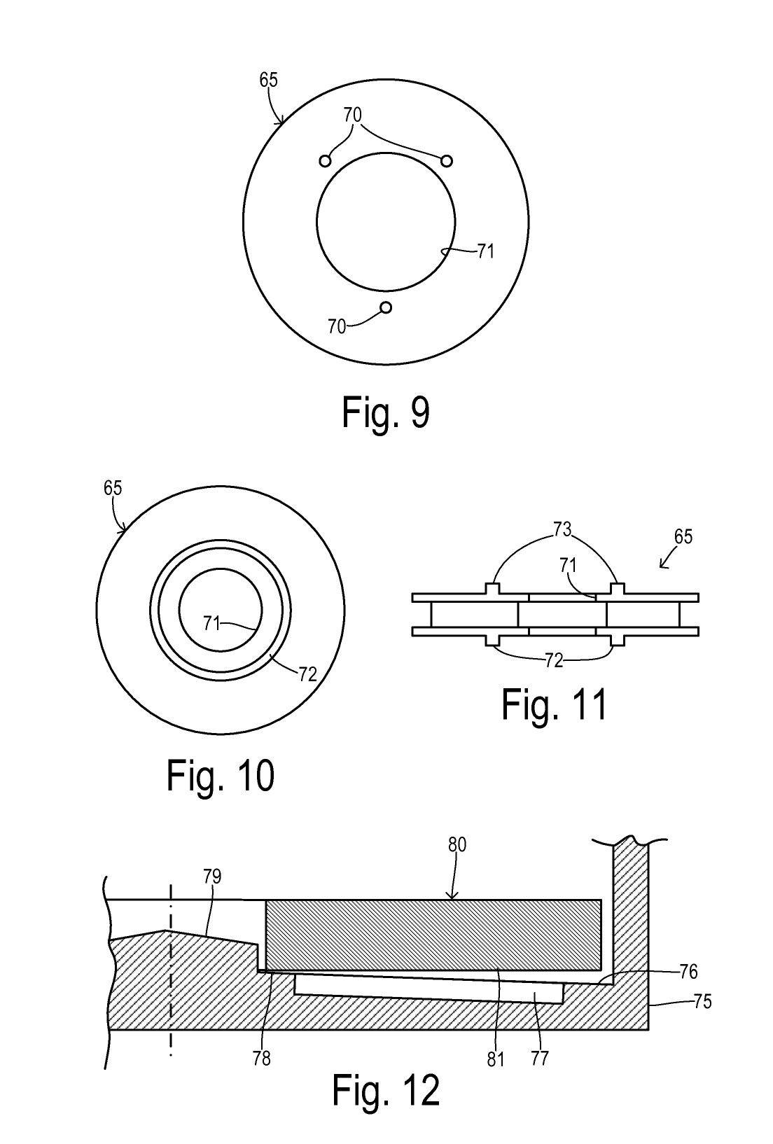

In order to achieve a smooth transition into a levitated condition when starting pump operation, a plurality of raised bumps may be placed symmetrically in order to maintain a parallel relationship between the impeller surfaces and the pump chamber walls. Thus, as shown in FIG. 9, impeller 65 may include a plurality of raised bumps 70 at different angular positions, each close to an inner edge 71 of impeller 65.

In an alternative embodiment shown in FIG. 10, a raised arcuate rib 72 is used instead of a plurality of raised bumps. Arcuate rib 72 may occupy a full 360° arc or may include rib sections with smaller arcs. Preferably, a similar arcuate rib 73 is disposed on the opposite side of impeller 65 as shown in FIG. 11.

In yet another embodiment as shown in FIG. 12, sloping walls can be utilized to maintain the desired separation between the impeller and the pump chamber walls. Thus, a pump housing 75 has a wall 76 containing hydrodynamic bearing features 77. An inner radial band 78 of wall 76 is free of features 77. A hub 79 receives impeller 80 which has a surface 81 juxtaposed with wall 76.

Wall 76 is arranged as a ramp surface so that radial band 78 provides an elevated edge of the ramp surface which receives a corresponding portion of surface 81 of impeller 80, thereby maintaining separation between bearing features 77 and surface 81. A very small contact region occurs at the inner radial edge of impeller 80 so that only a small amount of friction or abrasion is created.

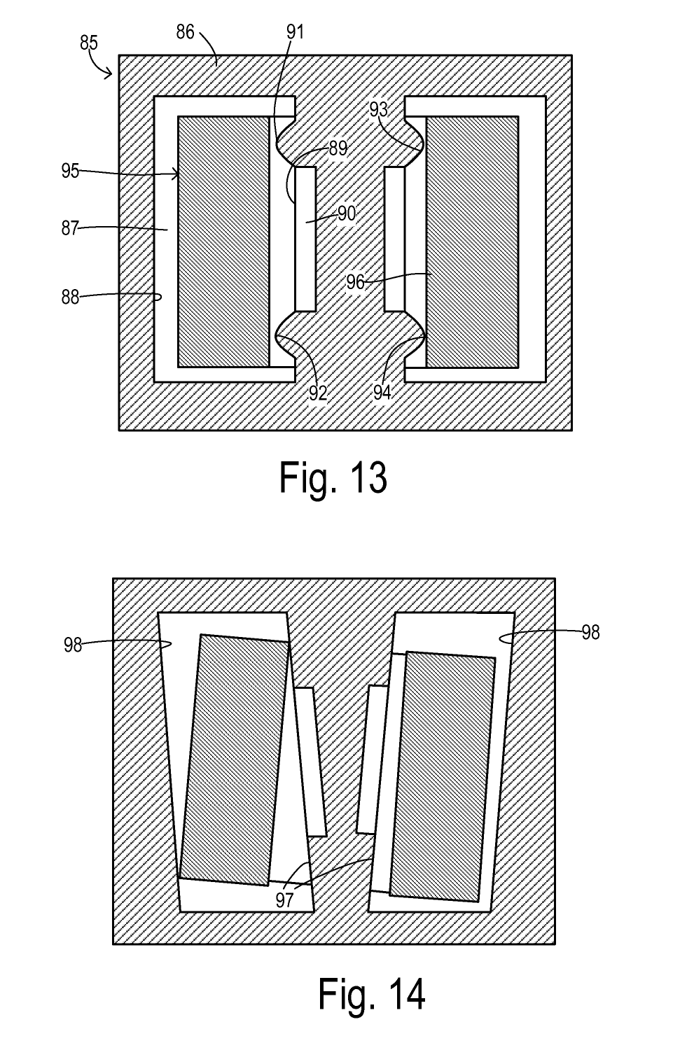

Besides the disc shaped impeller and pumping chamber shown above, the present invention can also be used with a cylindrically-shaped impeller and pumping chamber. As shown in FIG. 13, a pump 85 includes a pump housing 86 with a cylindrically-shaped pumping chamber 87 between an outside wall 88 and an inside wall 89. The inside wall 89 includes hydrodynamic bearing features 90 adapted to levitate an impeller 95 within pumping chamber 87 when rotating up to speed. Inside wall 89 includes mechanical thrust bearings in the form of a plurality of raised bumps 91-94 which maintain a predetermined separation between inner wall 89 and an inside surface 96 of impeller 95. Bumps 91 and 93 or 92 and 94 can be distinct bumps or may each be part of an arcuate rib extending along inner wall 89, for example.

FIG. 14 shows an alternative embodiment wherein an inside wall 97 and an outside wall 98 are each formed as ramped surfaces to provide elevated edges which prevent face-on contact between the impeller surfaces and the pump chamber walls.

In each of the foregoing embodiments, levitation of the impeller during normal impeller rotation is achieved in a conventional manner by directing a blood flow between the impeller and the pumping chamber walls to create hydrodynamic forces that urge the impeller into the levitated position. The mechanical thrust bearings are sufficiently small that they are not engaged when the impeller is at the levitated position and they do not significantly impact the normal blood flow. When stopped, the mechanical thrust bearings are engaged between the impeller and the chamber walls to maintain the predetermined separation between the hydrodynamic bearing features and the impeller. Thus, soft, biocompatible materials can be employed for the impeller and chamber walls such as titanium or titanium alloys.

What is claimed is:

1. A rotary blood pump comprising:
a pump housing with a pumping chamber between first and second walls; and
an impeller disposed in the pumping chamber, wherein the impeller is configured to operate in a levitated position spaced from the first and second walls in response to hydrodynamic forces that urge the impeller into the levitated position;
wherein a portion of the first and second walls includes hydrodynamic bearing features for increasing the hydrodynamic forces; and
wherein at least one of the impeller or the walls includes at least one mechanical thrust bearing extending between the impeller and each of the walls, wherein the mechanical thrust bearing is configured such that when the impeller is not being held in the levitated position by the hydrodynamic forces then the mechanical thrust bearing is engaged to maintain a predetermined separation between the hydrodynamic bearing features and the impeller, and wherein the mechanical thrust bearing is configured such that when the impeller is being held in the levitated position by the hydrodynamic forces then the mechanical thrust bearing is unengaged.

2. The pump of claim 1 wherein the mechanical thrust bearing is comprised of a raised bump.

3. The pump of claim 1 comprising a plurality of mechanical thrust bearings each comprised of a raised bump.

4. The pump of claim 3 wherein each of the walls includes a plurality of the raised bumps.

5. The pump of claim 3 wherein the plurality of raised bumps are located on the impeller for contacting each of the walls.

6. The pump of claim 1 wherein the mechanical thrust bearing is comprised of an arcuate rib.

7. The pump of claim 1 wherein the mechanical thrust bearing is comprised of an elevated edge of a ramp surface formed by one of the impeller or the first and second walls.

8. The pump of claim 1 wherein the impeller is substantially cylindrical with top and bottom surfaces juxtaposed with the first and second walls, respectively, so that the hydrodynamic bearing features act upon a primary radial band of the top and bottom surfaces, wherein the primary radial band occupies a majority of a total surface area of the top and bottom surfaces, and wherein the top and bottom surfaces have a secondary radial band that coincides with the mechanical thrust bearing.

9. The pump of claim 8 wherein the second radial band is radially inward from the primary radial band.

10. A method of supporting an impeller of a rotary blood pump disposed within a pump housing with a pumping chamber between first and second walls, comprising the steps of:
levitating the impeller within the pumping chamber during impeller rotation by directing a blood flow between the impeller and the first and second walls according to hydrodynamic bearing features formed in the first and second walls to create hydrodynamic forces that urge the impeller into the levitated position; and
engaging a mechanical thrust bearing between the impeller and one of the walls when the impeller is not rotating to maintain a predetermined separation between the hydrodynamic bearing features and the impeller, wherein the mechanical thrust bearing is configured such that when the impeller is being held in the levitated position by the hydrodynamic forces then the mechanical thrust bearing is unengaged.

11. The method of claim 10 wherein the mechanical thrust bearing is comprised of a raised bump.

12. The method of claim 10 comprising a plurality of mechanical thrust bearings each comprised of a raised bump.

13. The method of claim 12 wherein each of the walls includes a plurality of the raised bumps.

14. The method of claim 12 wherein the plurality of raised bumps are located on the impeller for contacting each of the walls.

15. The method of claim 10 wherein the mechanical thrust bearing is comprised of an arcuate rib.

16. The method of claim 10 wherein the mechanical thrust bearing is comprised of an elevated edge of a ramp surface formed by one of the impeller or the first and second walls.

17. The method of claim 10 wherein the impeller is substantially cylindrical with top and bottom surfaces juxtaposed with the first and second walls, respectively, so that the hydrodynamic bearing features act upon a primary radial band of the top and bottom surfaces, wherein the primary radial band occupies a majority of a total surface area of the top and bottom surfaces, and wherein the top and bottom surfaces have a secondary radial band that coincides with the mechanical thrust bearing.

18. The method of claim 17 wherein the second radial band is radially inward from the primary radial band.
